(19) United States
(12) Patent Application Publication
(10) Pub. No.: US 2012/0053383 A1
(43) Pub. Date: Mar. 1, 2012

(54) METHOD FOR PRODUCING OLEFINS BY DILUTE FEED CRACKING OF REFINERY OFF-GAS AND OTHER LIGHT HYDROCARBONS

(75) Inventors: Wadie MALATY, Houston, TX (US); Richard H. McCue, Houston, TX (US); David J. Brown, Ittre (BE); William Larson, Houston, TX (US)

(73) Assignee: Stone & Webster Process Technology, Inc., Houston, TX (US)

(21) Appl. No.: 13/217,716

(22) Filed: Aug. 25, 2011

Related U.S. Application Data
(60) Provisional application No. 61/376,755, filed on Aug. 25, 2010.

(51) Int. Cl. C07C 4/04; C07C 1/02; C10G 57/00

(52) U.S. Cl. 585/638; 208/95; 585/733; 585/752; 585/650; 585/648

(57) ABSTRACT

The present invention is directed to a method for producing, inter alia, olefins from refinery saturated and unsaturated off-gas. Furthermore, said refinery streams are not required to undergo deoxygenation reaction in a separate reactor system provided they are fed to the pyrolysis furnace. The refinery off-gases are treated to produce olefins such as ethylene and propylene. Gases from petrochemical facilities, gas separation plants and similar facilities that produce light gases containing ethane and propane are useful in the present method.

### CROSS-REFERENCE TO RELATED APPLICATIONS

This application claims priority to U.S. Provisional Patent Application No. 61/376,755 which was filed on Aug. 25, 2010 and is incorporated by reference as if fully set forth herein.

### FIELD OF THE INVENTION

The present invention relates generally to a method for the production of olefins. More particularly, the present invention is directed to a method for producing light olefins (e.g., ethylene, and propylene) and associated byproducts from refinery saturated and unsaturated off-gases and other light hydrocarbon feedstocks.

### BACKGROUND OF THE INVENTION

The petroleum refining and petrochemical industries have often sought new integration opportunities for refinery products with other processes. One of the areas of interest concerns refinery off-gases that are produced as a result of various separation and conversion processes, for example, crude distillation, fluid catalytic cracking, hydrocracking, hydrotreating, delayed coking, catalytic reforming, aromatics processing and the like. Many different off-gas streams containing mixtures of hydrogen and light hydrocarbons, such as C₁ to C₆ hydrocarbons, are generated during oil refining and petrochemical processing steps.

While these refinery off-gas streams are a potential source of hydrogen, ethane, propane and other compounds, many refinery and petrochemical off-gas streams are not used either for their hydrogen content or to generate other valuable compounds, e.g., olefins, due to a variety of economic and practical reasons. Some of the economical and practical reasons off-gas streams are not used include: the flow is too small, the pressure is too low, the content of valuable components is too low, or the contaminant level is too high. The off-gas streams are frequently consumed as fuel within the overall refinery/petrochemical complex.

Over the years, economic pressures have driven refiners to attempt to convert even the heaviest fractions of the crude oil to gasoline components and petrochemical feedstocks. For example, hydrocracking is widely used to break down aromatic cycle oils, coker distillates and other relatively heavy feeds and reconstitute them as diesel fuels, kerosene or naphtha. Separation of the components in the raw stream leaving the reactors is typically carried out by flashing off hydrogen and other gases, followed by various stripping and fractionation steps as appropriate. During these processes, however, considerable amounts of light hydrocarbon off-gas are not recaptured.

Representative refinery treatment reactor processes carried out in refineries or petrochemical plants that can give rise to off-gas streams (useful in the practice of the present invention) include, but are not limited to, catalytic cracking, catalytic reforming, delayed coking, distillate dewaxing, aromatics production, alkylation, isomerization, hydrocracking, hydrogenation, dehydrogenation, and olefin production. Other off-gas streams also arise from unsaturated and saturated gas plants used to treat and fractionate pooled off-gases from the various refinery fractionation or conversion units.

The result is the formation of a number of diverse streams from which it may not currently be cost effective to carry out further product recovery. Thus, these off-gases are frequently used as fuel within the overall complex. Higher profits can be realized if these off-gas streams could be efficiently processed to obtain higher value products. The recovery of olefins, such as ethylene and propylene from petro-chemical plant off-gas streams, however, is economically and environmentally important, but is a highly energy intensive process. Therefore, improved processes which can achieve this goal are of great interest.

As more fully disclosed herein, the present invention provides a method for utilizing off-gases from various refinery and petrochemical fractionation and conversion processes, as feeds to economically and practically produce olefins and other valuable compounds with little, or no initial pretreatment.

### SUMMARY OF THE INVENTION

The present invention provides a method for producing olefins in an integrated petrochemical facility comprised of at least one feedstock from a refinery unit, or other hydrocarbon processing unit and at least one downstream pyrolysis furnace. The method comprises: obtaining a refinery off-gas stream comprising at least one of ethane and propane from the upstream processing unit or units; combining the off-gas stream(s) with a pyrolysis furnace ethane or propane feed stream and/or any other conventional cracking furnace feedstock and saturating the combined stream with dilution steam in a feed saturator or mixing it with dilution steam. The method continues by cracking the combined stream in the downstream pyrolysis furnace to produce cracked product, and separating the cracked product into one or more of hydrogen, methane, ethylene, propylene, butenes, heavier products, a fuel gas stream and recycle streams in the unit recovery systems.

The inventive method allows the upgrade of the ethane, propane and other hydrocarbons contained in the refinery off-gas to more valuable cracking feedstock without significant investment in compression and pre-fractionation processes. The contained lighter gases, mainly hydrogen and methane, act as diluents to lower the hydrocarbon partial pressure, which improves the yield selectivity to the desired ethylene with only a slight reduction in propylene. Moreover, the benefits of the present invention can be achieved in conjunction with higher coil outlet pressures, for example, 2.4-2.8 bara (35-40 psia) and/or lower steam to hydrocarbon ratios in the range of 0.1 to 0.3 and most preferably 0.15 to 0.2, while achieving optimum yield and energy efficiency.

Further, the present invention can eliminate or significantly reduce compression, refrigeration and fractionation of the contained light gases, such as, hydrogen and methane from ethane and heavier feeds prior to cracking of said ethane, propane and heavier feeds. The contained oxygen will be converted completely to carbon monoxide, carbon dioxide and water without the need to invest in separate oxygen removal facilities.

### BRIEF DESCRIPTION OF THE DRAWINGS

FIG. 1 is a schematic drawing showing Prior Art of the method of producing olefins from the off-gases of an integrated petrochemical facility.

FIG. 2 is a schematic drawing showing of the present inventive method for producing olefins from the off-gases of an integrated petrochemical facility.

### DETAILED DESCRIPTION OF THE PRESENT INVENTION

All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. Thus, the scope of the invention should be determined by the appended claims and their equivalents, rather than by the examples given.

Whenever a range is given in the specification, for example, a temperature range, a time range, a flow-rate range, or a size range, all intermediate ranges and subranges, as well as all individual values included in the ranges given are intended to be included in the disclosure.

The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general, the terms and phrases used herein have their art-recognized meaning, which can be found by reference to standard texts, journal references and contexts known to those skilled in the art. The following definitions are provided to clarify their specific use in the context of the invention.

For purposes of this invention, the phrase “refinery treatment reactor” means any one of the typical petrochemical process units in a petrochemical refinery. Examples of refinery treatment reactors include, crude distillation units (i.e., atmospheric distillation, vacuum distillation unit), naphtha hydrotreater unit, catalytic reformer unit, distillate hydrotreater unit, fluid catalytic cracker (FCC) unit, hydrocracker unit, visbreaking unit, merox unit, coking units (i.e., delayed coking, fluid coker, and flexicoker), alkylation unit, dimerization unit, isomerization unit, aromatics units, steam reforming unit, amine gas treater, and claus units.

As used herein the term “off-gas” means a refinery treatment reactor product, by-product or waste gas stream that is produced from any one of the aforementioned refinery treatment reactor processes or any other petrochemical or gas processing off-gas stream.

The present invention relates to a unique method for producing olefins in an integrated petrochemical facility from off-gas streams from refinery processing units. Olefins find widespread uses in many industries. For example, they represent the basic building blocks in such diverse uses as film and packaging, communications, construction, automotive and home appliances. These important materials are generally produced by the cracking of a hydrocarbon feedstream, which converts saturated hydrocarbons present in the feedstream into olefins, or the recovery of light olefins from unsaturated streams such as FCC off-gas.

The inventive method utilizes refinery saturated and nearly saturated off-gas streams from refinery treatment reactor(s) and, with or without further processing, directs the off-gas stream to a pyrolysis process to produce olefins and other valuable products.

The off-gas streams for the method of the present invention typically comprise, but are not limited to, hydrogen, carbon monoxide, carbon dioxide, methane, acetylene, ethylene, ethane, methyl acetylene, propadiene, propylene, propane, butadienes, butanes, butenes and heavier C₅₊ hydrocarbons. The off-gas streams can also include light olefin components, typically C₂ to C₅ olefins, although olefins with higher carbon numbers may also be used. Sources of the off-gas streams normally include: light gas streams recovered from the gas separation section of a refinery fluid catalytic cracking (FCC) process, a sweet refinery gas, coker off-gas, effluents from light paraffin (e.g. LPG) dehydrogenation zones, saturated gas separation unit or other type of off-gas containing high amounts of hydrocarbons with more than two carbon atoms.

The off-gas stream may contain oxygen, nitrogen and other contaminants such as, but not limited to hydrogen sulfide and carbon dioxide. As such, the gases from petrochemical facilities, gas separation plants, and similar facilities, which are well known in the art and produce light gases are useful as off-gas streams in the present invention.

The refinery treatment reactor off-gas stream of the presently claimed method is sent directly to at least one downstream pyrolysis cracking furnace. The inventive method eliminates or greatly reduces the need for currently practiced processes, such as, compression, refrigeration and pre-fractionation, of off-gas streams prior to downstream pyrolytic cracking. The off-gas by itself, or combined with a typical ethane or propane feed is sent to a pyrolysis system without further fractionation.

The downstream pyrolysis furnace may be any type of conventional pyrolysis furnace, especially including a tubular steam cracking furnace, designed for pyrolizing light and/or heavy feed and operated for production of lower boiling products such as olefins. Examples of pyrolysis furnaces useful in the present invention include those disclosed in the following U.S. Pat. Nos. 3,487,121 to Hallee, 3,972,682 to Stephens et al., 4,020,273 to Dix et al., 4,765,883 to Johnson et al., 5,181,990 to Arisaki et al., 5,271,827 to Woebcke and 6,419,885 to Di Nicolantonio et al. The contents of each of the above-referenced patents are incorporated herein by reference for all purposes.

The off-gas, or off-gases, are directed to the downstream pyrolysis cracking furnace(s) at any suitable conditions that provide the necessary cracking to the desired olefinic compound product(s). Accordingly, the off-gas is directed to the downstream pyrolysis cracking furnace(s) at pressures ranging from about 4 bara to about 12 bara. As such, the pressure ranges correspond to a furnace coil outlet pressure that will typically be in the range of about 1.2 bara to about 2.8 bara but could also be as high as about 5 bara or as low as about 1 bara to produce primarily light olefins, e.g., ethylene and propylene.

The pyrolysis furnace feed includes dilution steam, which is generated separately and added to the refinery treatment reactor(s) off-gas, ethane, propane, and other selected furnace feeds. Likewise, the refinery treatment reactor(s) off-gas can be humidified in a saturator system. Dilution steam systems and saturator systems for cracking hydrocarbons are well known in the art. For example, U.S. Pat. Nos. 3,487,121 to Hallee and 4,940,828 to Pettersen et al. disclose dilution steam for cracking hydrocarbons, the entire contents of which are incorporated herein by reference. The cracking will occur in the presence of dilution steam typically in the range of about 0.1 to about 0.4 on a steam to feed weight basis, however, the steam to feed weight basis can be as low as 0 or as high as about 0.7.

Once the off-gas stream is introduced into the downstream pyrolysis cracking furnace, the cracking reactions can take place at any suitable conditions that provide the necessary cracking to the desired olefinic compound product(s). Generally, the cracking temperature of the furnace can be in a range of from about 1000° F. to about 2000° F., preferably about 1100° F. to about 1850° F., and most preferably 1250° F. to 1650° F. The residence time of the hydrocarbon fluid, based on the conditions described above, is generally in the range of from about 0.02 second to about 0.5 second, more preferably from about 0.02 to about 0.2 seconds.

The time required for converting a saturated hydrocarbon to an olefinic compound can vary widely depending on the hydrocarbon used in the process, the olefinic compound(s) desired, and the rate of the introduction of off-gas stream. Generally, the flow rate of the off-gas stream is in the range of from about 6,000 to about 20,000 pounds per hour per cracking coil depending on the capacity of the cracking furnace.

Also, useful in the method of the present invention are pyrolysis furnace feeds following limited pre-fractionation such as, but not limited to, deethanization (e.g., in a deethanization column as known in the art) to allow separate cracking of the ethane-rich gas, propane and heavier components of the feed to achieve optimum olefin yield. Optionally, the method provides limited contaminant removal from the pyrolysis furnace feed. A non-limiting example of contaminant removal would include amine treatment to remove acid gas, such as hydrogen sulfide and carbon dioxide. Most significant of the inventive method is that oxygen contained in the furnace feed is completely converted to carbon monoxide, carbon dioxide and water, thus elimination the need for the deoxygenation reactor system.

Once the off-gas stream has been subjected to downstream pyrolysis, known and conventional processes are used to separate the mainly C₁ to C₃ gaseous mixtures containing large amounts of ethene (ethylene), ethane, propylene, propane and methane are performed. In addition contained C₄₊ components are also fractionated. Significant amounts of hydrogen usually accompany cracked hydrocarbon gas, along with minor amounts of acetylene. The acetylene component may be removed before or after cryogenic operations (see, e.g., U.S. Pat. No. 5,414,170 to McCue et al.). The cracked off-gas stream typically is compressed at ambient temperature or below and at process pressure of at least about 2500 kPa (350 psig), preferably about 3700 kPa (37.1 kgf/cm², 520 psig), then separated in a chilling train under cryogenic conditions into several liquid streams and gaseous methane/hydrogen streams. The more valuable olefin streams are decontaminated prior to recovery.

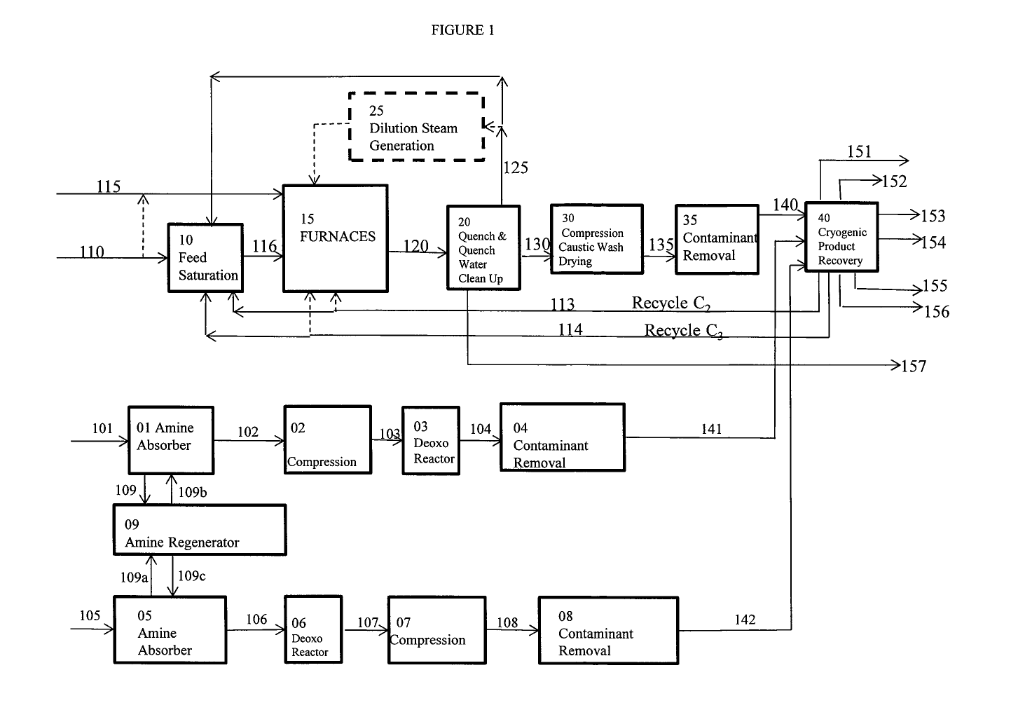

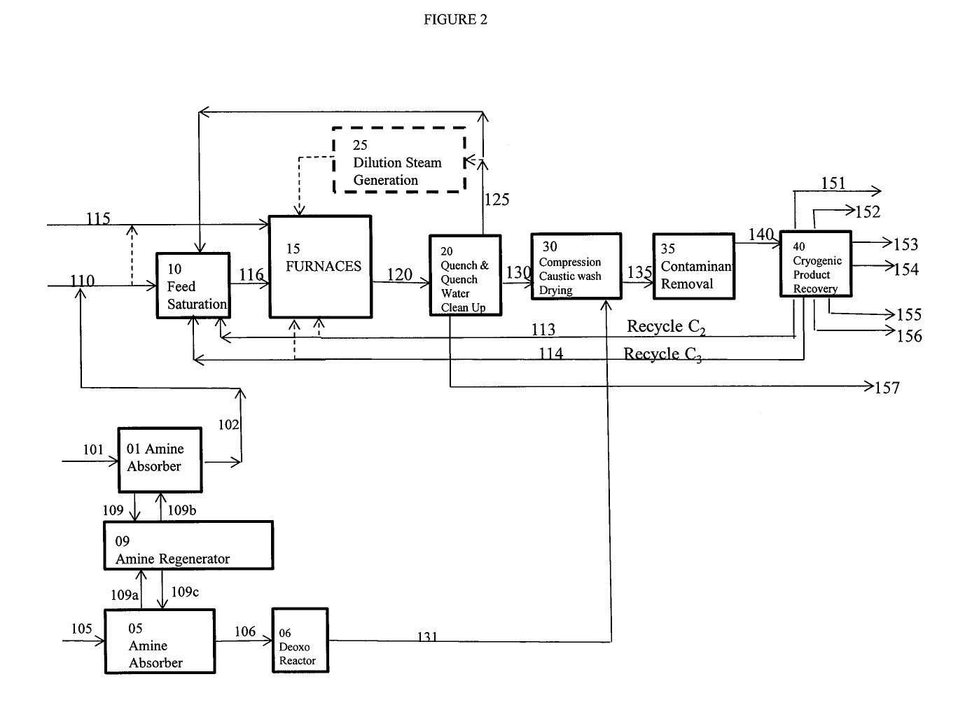

While the specification concludes with claims distinctly pointing at the subject matter that applicants regards as their invention, it is believed that the invention will be better understood when taken in connection with the accompanying FIGS. 1 and 2, which illustrate the schematic of a preferred embodiment for carrying out a method in accordance with the present invention.

In FIGS. 1 and 2, the saturated or nearly saturated refinery off-gases, or other light hydrocarbon feed (stream 101) are obtained from, for example, a coking unit and saturated refinery gas unit. The unsaturated refinery off-gases are obtained from, for example, a fluid catalytic cracker unit (Stream 105), however, the off-gas can be obtained from any saturated and unsaturated off-gases produced by a refinery treatment reactor. With reference to the FIGS. 1 and 2, the saturated (or nearly saturated) off-gas stream 101 is routed to amine absorber unit 01 operating at a temperature of from about 90° F. to about 130° F. and a pressure ranging from about 120 psig to about 300 psig. The amine absorber 01 is operatively connected to an amine regenerator unit 09 to regenerate the amine absorbent used in amine absorber 01 to remove acid gas from the off-gas feed stream 101, which is operatively connected via 109 (i.e., rich amine from saturated gas absorber to amine regenerator) and 109b (i.e., lean amine from amine regenerator to saturated gas amine absorber), as is well known to those skilled in the art.

Unsaturated refinery gas, from, for example, FCC gas, in stream 105 is directed to amine absorber 05, which is also operatively connected to an amine regenerator 09, via 109a (i.e., rich amine from unsaturated gas absorber to amine regenerator) and 109c (i.e., lean amine from amine regenerator to unsaturated gas amine absorber). The effluent from the amine absorber 05 in a line 106 is directed to a selective deoxygenation reactor 06 for conversion of oxygen to water and nitrogen oxide to ammonia and water.

In the embodiment of prior art in FIG. 1, the amine treated saturated (or nearly saturated) off-gas in stream 102 is compressed in a multistage compressor system unit 02 to a pressure ranging from about 300 psig to about 550 psig where in the saturated off-gas stream 103 (i.e., saturated or nearly saturated off-gas from compression to deoxygenation) is routed to a deoxygenation reactor 03 which could be located in an intermediate compression stage or at the discharge of the final compression stage for oxygen conversion. The effluent from the deoxygenation reactor stream 104 (i.e., saturated or nearly saturated off-gas from deoxygenation to contaminant removal) flows to contaminant removal 04 which includes drying. The dried and treated saturated gas stream 141 (i.e., saturated or nearly saturated off-gas from contaminant removal to cryogenic recovery), flows to a separate or combined cryogenic product recovery section 40.

In the embodiment of prior art in FIG. 1, the unsaturated gas effluent from the deoxygenation reactor system 06, via 107 (i.e., unsaturated off-gas from deoxygenation to compression), is compressed in a single or multistage compressor system unit 07 to a pressure ranging from about 300 psig to about 550 psig where in the unsaturated off-gas stream, via 108 (i.e., unsaturated off-gas from compression to contaminant removal), is routed to contaminant removal unit 08, which includes drying. The dried and treated unsaturated gas stream flows, via 142 (i.e., unsaturated off-gas from contaminant removal to cryogenic recovery), to a separate or combined cryogenic product recovery section 40.

In an embodiment of the claimed method of FIG. 2, the unsaturated gas effluent from the deoxygenation reactor system 06, via 131 (i.e., unsaturated gas from the deoxygenation to cracked gas compression), is routed to main cracked gas compression, caustic wash, and drying section 30. This eliminates the need for separate compression and contaminant removal as embodied in prior art FIG. 1, i.e., compression system 07, and contaminant removal 08.

In the embodiment of prior art in FIG. 1, traditional pyrolysis liquid feeds (containing C₄, C₅, etc.) 115 (i.e., traditional pyrolysis liquid feed), and traditional pyrolysis gas feeds (C₂, C₃ etc.) 110 (i.e., traditional pyrolysis gas feed), are fed to the pyrolysis section which includes feed saturation unit 10 and/or dilution steam generation 25 |[WM1], and the pyrolysis furnaces 15. Process water, via 125, to dilution steam generator or saturator |[WM2]

In a specific embodiment of the claimed method of FIG. 2, the amine treated saturated off-gas in a line 102 (i.e., saturated or nearly saturated off-gas from amine absorber), is routed directly to the pyrolysis section which includes gas feed saturation unit 10 and/or dilution steam generation 25 and the pyrolysis furnaces 15. This eliminates the need for separate saturated gas compression, deoxygenation and contaminant removal as embodied in prior art FIG. 1, i.e., saturated or nearly saturated compressor system 02, saturated or nearly saturated deoxygenation reactor 03 and saturated or nearly saturated gas contaminant removal unit 04.

As such, the inventive method eliminates the prior art steps of compression 02, deoxygenation 03 and the remaining contaminant removal 04 as presented in FIG. 1. The presently claimed method directs the amine treated 01 saturated (or nearly saturated) gases shown as stream 102 in FIG. 1, and routes it directly to the feed 110 of the pyrolysis furnaces as presented in FIG. 2. Additionally, separate unsaturated gas compression 07 and unsaturated gas contaminant removal 08 as presented in FIG. 1 are eliminated for the unsaturated gases, for example, FCC gas.

In known and conventional manner as depicted in both FIGS. 1 and 2, furnace effluent stream 120 (i.e., combined furnace effluent) is quenched in quench and quench water cleanup unit 20, and then stream 130 (i.e., cracked gas from quench to compression) is compressed in cracked gas compression, caustic wash and drying unit 30. In unit 30 the cracked gas also undergoes acid gas removal in a caustic wash section and dried under conditions known to those of ordinary skill in the art.

In known and conventional manner as depicted in both FIGS. 1 and 2, compressed effluent undergoes contaminant removal via 135 in cracked gas contaminant removal unit 35. Final products are separated in cryogenic product recovery unit 40, via 140 (i.e., cracked gas to cryogenic product recovery) under conditions known to those of ordinary skill in the art. Recycled ethane via 113 and recycled propane via 114 are routed to the furnaces 15 via gas feed saturation 10 and saturated gas feed to furnaces 116 or directly to the furnaces 15. From cryogenic product recovery unit 40 there is recovered: hydrogen product via 151; fuel gas product via 152; polymer grade ethylene product via 153; polymer (or chemical) grade propylene product via 154; raw (or hydrogenated) C₄ product via 155; raw (or hydrogenated) pyrolysis gasoline product via 156; from Product Recovery; and pyrolysis fuel oil product from quench and quench water clean-up unit 20.

While the present invention has been described with reference to a preferred embodiment, as will occur to those skilled in the art, numerous changes, additions and omissions may be made without departing from the spirit and scope of the present invention.

A material balance for the method presented in FIG. 2 is given in the tables of the following prophetic Examples. All units are based on steady state continuous stream conditions.

TABLE 1: Presents an exemplary overall material balance feed useful in the method of the present invention.

TABLE 1

| Ethylene Plant | |
|---|---|
| S/O C2 | 0.15 |
| S/O C3 | 0.3 |
| TLX Pout, psia | 35.7 |

| | kg/hr | kTa |
|---|---|---|
| Feeds | | |
| Total Sat Gas | 155933.8 | 1309.844 |
| Coker Gas to Furnaces | 90762 | 762.4008 |
| Refinery C3 To Furnaces | 65476.19 | 550 |
| FCC Gas to Recovery | 134968 | 1133.731 |
| Total | 447140 | 3755.976 |
| Products | | |
| C4 + to Refinery | 23652 | 198.6768 |
| Hydrogen Product | 14690 | 123.396 |
| Residue Gas to Fuel | 159936 | 1343.462 |
| Ethylene Product | 192976 | 1620.998 |
| Propylene Product | 33942 | 285.1128 |
| Raw C₄ Product | 9733 | 81.7572 |
| Raw Pyrolysis Gasoline | 10878 | 91.3752 |
| Fuel Oil | 1333 | 11.1972 |
| Total | 447140 | 3755.976 |

TABLE 2: Presents Prophetic Examples of upstream off-gas feeds from refinery treatment reactors useful in the present method of producing olefins as an ethane rich feed.

TABLE 2
Feed to Ethane Furnaces

| | | | | | Example 1: | | | | | |
|---|---|---|---|---|---|---|---|---|---|---|
| | | | | | Saturated Refinery | | Example 2: | | Example 3: | |
| | | Total | | | Off-Gas | | Coker Off-Gas | | Ethane Recycle | |
| | | kg/hr | mols/hr | wt % | kg/hr | wt % | kg/hr | wt % | kg/hr | wt % |
| 1 | HYDROGEN | 10428 | 5172.619 | 3.53 | 9431 | 9.87 | 997 | 1.1 | 0 | 0 |
| 2 | METHANE | 57184 | 3564.421 | 19.36 | 14596 | 15.27 | 42588 | 46.92 | 0 | 0 |
| 3 | CO | 469 | 16.74342 | 0.16 | 0 | 0 | 469 | 0.52 | 0 | 0 |
| 4 | ACETYLENE | 0 | 0 | 0 | 0 | 0 | 0 | 0 | 0 | 0 |
| 5 | ETHYLENE | 5524 | 196.906 | 1.87 | 0 | 0 | 4507 | 4.97 | 1017 | 0.93 |
| 6 | ETHANE | 212219 | 7057.499 | 71.85 | 68863 | 72.05 | 35672 | 39.3 | 107684 | 98.79 |
| 7 | MACETYLENE | 0 | 0 | 0 | 0 | 0 | 0 | 0 | 0 | 0 |
| 8 | PROPDIENE | 0 | 0 | 0 | 0 | 0 | 0 | 0 | 0 | 0 |
| 9 | PROPYLENE | 1810 | 43.01331 | 0.61 | 3 | 0 | 1502 | 1.65 | 305 | 0.28 |
| 10 | PROPANE | 2390 | 54.1987 | 0.81 | 618 | 0.65 | 1772 | 1.95 | 0 | 0 |
| 11 | 1,3 BUTADIENE | 0 | 0 | 0 | 0 | 0 | 0 | 0 | 0 | 0 |
| 12 | 1 BUTENE | 0 | 0 | 0 | 0 | 0 | 0 | 0 | 0 | 0 |
| 13 | ISOBUTENE | 1259 | 22.43887 | 0.43 | 0 | 0 | 1259 | 1.39 | 0 | 0 |
| 14 | CIS 2BUTENE | 0 | 0 | 0 | 0 | 0 | 0 | 0 | 0 | 0 |
| 15 | TRANS 2BUTENE | 0 | 0 | 0 | 0 | 0 | 0 | 0 | 0 | 0 |
| 16 | Iso BUTANE | 46 | 0.791411 | 0.02 | 0 | 0 | 46 | 0.05 | 0 | 0 |
| 17 | BUTANE | 782 | 13.45399 | 0.26 | 0 | 0 | 782 | 0.86 | 0 | 0 |
| 18 | 1,3 CYCLOPNTDN | 0 | 0 | 0 | 0 | 0 | 0 | 0 | 0 | 0 |
| 19 | ISOPRENE | 0 | 0 | 0 | 0 | 0 | 0 | 0 | 0 | 0 |
| 20 | C13PENTADIENE | 0 | 0 | 0 | 0 | 0 | 0 | 0 | 0 | 0 |
| 21 | 1 PENTENE | 0 | 0 | 0 | 0 | 0 | 0 | 0 | 0 | 0 |
| 22 | Iso PENTANE | 0 | 0 | 0 | 0 | 0 | 0 | 0 | 0 | 0 |
| 23 | PENTANE | 0 | 0 | 0 | 0 | 0 | 0 | 0 | 0 | 0 |
| 24 | 1 HEXENE | 38 | 0.451505 | 0.01 | 0 | 0 | 38 | 0.04 | 0 | 0 |
| 25 | BENZENE | 96 | 1.228957 | 0.03 | 0 | 0 | 96 | 0.11 | 0 | 0 |
| 26 | TOLUENE | 96 | 1.04187 | 0.03 | 0 | 0 | 96 | 0.11 | 0 | 0 |
| 27 | OXYLENE | 38 | 0.35792 | 0.01 | 0 | 0 | 38 | 0.04 | 0 | 0 |
| 28 | C9-200C | 57 | 0.471074 | 0.02 | 0 | 0 | 57 | 0.06 | 0 | 0 |
| 29 | FUEL OIL | 57 | 0.341317 | 0.02 | 0 | 0 | 57 | 0.06 | 0 | 0 |
| 30 | N2 | 2854 | 101.8798 | 0.97 | 2068 | 2.16 | 786 | 0.87 | 0 | 0 |
| | Total | 295346 | | 100 | 95579 | 100 | 90762 | 100 | 109005 | 100 |

TABLE 3: Presents Prophetic Examples of off-gas feeds from ‘fresh’ refinery propane, propane rich stream from a saturated refinery gas, and refinery recycled propane, all of which are useful in the present method for producing olefins.

TABLE 3
Feed to Propane Rich Furnaces

| | | | | Example 4: | | Example 6: | | | |
|---|---|---|---|---|---|---|---|---|---|
| | | | | Fresh Refinery | | C₃ Rich Stream from | | Example 5: | |
| | | Total | | Propane | | Sat Refinery Gas | | Propane Recycle | |
| | | kg/hr | wt % | kg/hr | wt % | kg/hr | wt % | kg/hr | wt % |
| 1 | HYDROGEN | 0 | 0.00 | 0 | 0.00 | 0 | 0.00 | 0 | 0.00 |
| 2 | METHANE | 0 | 0.00 | 0 | 0.00 | 0 | 0.00 | 0 | 0.00 |
| 3 | CO | 0 | 0.00 | 0 | 0.00 | 0 | 0.00 | 0 | 0.00 |
| 4 | ACETYLENE | 0 | 0.00 | 0 | 0.00 | 0 | 0.00 | 0 | 0.00 |
| 5 | ETHYLENE | 0 | 0.00 | 0 | 0.00 | 0 | 0.00 | 0 | 0.00 |
| 6 | ETHANE | 3301 | 2.36 | 655 | 1.00 | 2646 | 7.21 | 0 | 0.00 |
| 7 | MACETYLENE | 7 | 0.01 | 0 | 0.00 | 0 | 0.00 | 7 | 0.02 |
| 8 | PROPDIENE | 0 | 0.00 | 0 | 0.00 | 0 | 0.00 | 0 | 0.00 |
| 9 | PROPYLENE | 4423 | 3.17 | 2619 | 4.00 | 13 | 0.04 | 1791 | 4.78 |
| 10 | PROPANE | 129985 | 93.10 | 60893 | 93.00 | 33447 | 91.13 | 35645 | 95.19 |
| 11 | 1,3 BUTADIENE | 2 | 0.00 | 0 | 0.00 | 0 | 0.00 | 2 | 0.01 |
| 12 | 1 BUTENE | 0 | 0.00 | 0 | 0.00 | 0 | 0.00 | 0 | 0.00 |
| 13 | ISOBUTENE | 0 | 0.00 | 0 | 0.00 | 0 | 0.00 | 0 | 0.00 |
| 14 | CIS 2BUTENE | 0 | 0.00 | 0 | 0.00 | 0 | 0.00 | 0 | 0.00 |
| 15 | TRANS 2BUTENE | 0 | 0.00 | 0 | 0.00 | 0 | 0.00 | 0 | 0.00 |
| 16 | Iso BUTANE | 1517 | 1.09 | 982 | 1.50 | 535 | 1.46 | 0 | 0.00 |
| 17 | BUTANE | 389 | 0.28 | 327 | 0.50 | 62 | 0.17 | 0 | 0.00 |
| 18 | 13CYCLOPNTDN | 0 | 0.00 | 0 | 0.00 | 0 | 0.00 | 0 | 0.00 |
| 19 | ISOPRENE | 0 | 0.00 | 0 | 0.00 | 0 | 0.00 | 0 | 0.00 |
| 20 | C13PNTDN | 0 | 0.00 | 0 | 0.00 | 0 | 0.00 | 0 | 0.00 |
| 21 | 1 PENTENE | 0 | 0.00 | 0 | 0.00 | 0 | 0.00 | 0 | 0.00 |
| 22 | Iso PENTANE | 0 | 0.00 | 0 | 0.00 | 0 | 0.00 | 0 | 0.00 |
| 23 | PENTANE | 0 | 0.00 | 0 | 0.00 | 0 | 0.00 | 0 | 0.00 |
| 24 | 1 HEXENE | 0 | 0.00 | 0 | 0.00 | 0 | 0.00 | 0 | 0.00 |
| 25 | BENZENE | 0 | 0.00 | 0 | 0.00 | 0 | 0.00 | 0 | 0.00 |
| 26 | TOLUENE | 0 | 0.00 | 0 | 0.00 | 0 | 0.00 | 0 | 0.00 |
| 27 | OXYLENE | 0 | 0.00 | 0 | 0.00 | 0 | 0.00 | 0 | 0.00 |
| 28 | C9-200C | 0 | 0.00 | 0 | 0.00 | 0 | 0.00 | 0 | 0.00 |
| 29 | FUEL OIL | 0 | 0.00 | 0 | 0.00 | 0 | 0.00 | 0 | 0.00 |
| | Total | 139624 | 100.00 | 65476.19 | 100.00 | 36702 | 100.00 | 37445 | 100.00 |

TABLE 4: Presents Prophetic Examples of the present method effluent and recovered products.

TABLE 4
Composite Furnace Effluents

| | Example 7: | | Example 8: | |
|---|---|---|---|---|
| | Total C2 Furnace | | Total C3 Furnace | |
| | Effluent | | Effluent | |
| Key Component | Ethane | | Propane | |
| Conversion, % of | 65 | | 74 | |
| key component | | | | |
| DS/HC, lb/lb | 0.15 | | 0.3 | |
| TLX Outlet | 35.7 | | 35.7 | |
| Pressure, psia | | | | |
| | kg/hr | wt % | kg/hr | wt % |
| HYDROGEN | 17906 | 6.062638 | 1705 | 1.221163 |
| METHANE | 72443 | 24.52785 | 23172 | 16.59636 |
| CO | 683 | 0.231251 | 27 | 0.019338 |
| ACETYLENE | 862 | 0.291857 | 301 | 0.215584 |
| ETHYLENE | 117974 | 39.9438 | 37109 | 26.57838 |
| ETHANE | 74252 | 25.14034 | 6989 | 5.005694 |
| MACETYLENE | 57 | 0.019299 | 109 | 0.078068 |
| PROPDIENE | 57 | 0.019299 | 76 | 0.054433 |
| PROPYLENE | 1602 | 0.542407 | 27446 | 19.6575 |
| PROPANE | 256 | 0.086677 | 33826 | 24.22701 |
| 1,3 BUTADIENE | 2219 | 0.751312 | 2078 | 1.488315 |
| 1 BUTENE | 294 | 0.099543 | 1013 | 0.725536 |
| ISOBUTENE | 20 | 0.006772 | 345 | 0.247097 |
| CIS 2BUTENE | 50 | 0.016929 | 170 | 0.121758 |
| TRANS 2BUTENE | 81 | 0.027425 | 217 | 0.155421 |
| Iso BUTANE | 0 | 0 | 76 | 0.054433 |
| BUTANE | 804 | 0.272219 | 309 | 0.221313 |
| 13CYCLOPNTDN | 365 | 0.123582 | 883 | 0.632426 |
| ISOPRENE | 0 | 0 | 18 | 0.012892 |
| C13PNTDN | 129 | 0.043677 | 461 | 0.33018 |
| 1 PENTENE | 79 | 0.026748 | 142 | 0.101704 |
| Iso PENTANE | 0 | 0 | 0 | 0 |
| PENTANE | 0 | 0 | 5 | 0.003581 |
| 1HEXENE | 100 | 0.033858 | 388 | 0.277895 |
| BENZENE | 1417 | 0.47977 | 1573 | 1.126621 |
| TOLUENE | 163 | 0.055189 | 357 | 0.255692 |
| OXYLENE | 116 | 0.039275 | 314 | 0.224895 |
| C9-200C | 77 | 0.026071 | 174 | 0.124623 |
| FUEL OIL | 492 | 0.166582 | 338 | 0.242084 |
| N2 | 2852 | 0.965634 | 0 | 0 |
| Total | 295350 | 100 | 139621 | 100 |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A method for producing olefins in an integrated petrochemical facility comprising at least one upstream feedstock refinery treatment reactor and at least one downstream pyrolysis furnace, said method comprising:
a) obtaining an off-gas stream comprising at least one of ethane and propane from said upstream feedstock refinery treatment reactor;
b) combining said off-gas stream with a pyrolysis furnace ethane, propane or other feed stream and saturating said combined stream with dilution steam;
c) cracking said combined stream in the downstream pyrolysis furnace to produce cracked product; and
d) separating said cracked product into one or more of hydrogen, methane, ethylene, propylene, heavier products and a fuel stream.

2. The method of claim 1 wherein the off-gas stream is at least one obtained from a refinery distillation unit, a refinery naphtha hydrotreater unit, a refinery catalytic reformer unit, a refinery distillate hydrotreater unit, a refinery fluid catalytic cracker unit, a refinery hydrocracker unit, a refinery visbreaking unit, a refinery merox unit, a refinery coking unit, a refinery alkylation unit, a refinery dimerization unit, a refinery isomerization unit, a refinery steam reforming unit, a refinery amine gas treater, and a refinery claus units.

3. The method of claim 1 wherein the off-gas stream comprises at least one of the compounds selected from the group consisting of hydrogen, carbon monoxide, carbon dioxide, methane, acetylene, ethylene, ethane, methyl acetylene, propadiene, propylene, propane, butadienes, butanes, butenes, benzene, and toluene.

4. The method of claim 1 further comprising subjecting said off-gas stream from said upstream feedstock refinery treatment reactor to a separation process to separate C₂ and lighter constituents from C₃ and heavier constituents to produce a light ends stream and a heavy ends stream.

5. The method of claim 1 wherein the pyrolysis furnace ethane and propane feed comprises ethane and propane obtained from said refinery treatment reactor and/or recycled ethane and propane and/or any other conventional cracking furnace feedstock.

6. The method of claim 1 wherein said pyrolysis furnace is at least one pyrolysis furnace for cracking ethane, or propane.

7. The method of claim 6 wherein the pyrolysis furnace for cracking ethane has a cracking reaction zone temperature from about 1000° to 2000° F.

8. The method of claim 6 wherein the pyrolysis furnace for cracking propane has a cracking reaction zone temperature from about 1000° to 2000° F.

9. The method of claim 1 wherein the combined stream is charged directly to said downstream pyrolysis furnace.

10. The method of claim 1 wherein the off-gas is charged to the downstream pyrolysis cracking furnace at pressures ranging from about 4 bara to about 12 bara.

11. The method of claim 1 wherein dilution steam is present in an amount that ranges from 0.0 to 0.7 on a steam to feed weight basis.

12. The method of claim 1 further comprising a pre-fractionation means for said off-gas stream.

13. The method of claim 12 wherein the pre-fractionation means comprises deethanizing the off-gas stream.

14. The method of claim 12 wherein the off-gas stream is a saturated refinery gas.

15. The method of claim 1 wherein once the off-gas stream has been subjected to downstream pyrolysis the off-gas is further subjected to a separation process to separate C₂ and lighter constituents from C₃ and heavier constituents to produce a light ends stream and a heavy ends stream.
